(19) United States
(12) Patent Application Publication
(10) Pub. No.: US 2026/0348987 A1
(43) Pub. Date: Nov. 5, 2026

(54) MINI-SCALE EUGLOBULIN CLOT LYSIS ASSAYS AND METHODS OF USING THE SAME

(71) Applicant: THE JOHNS HOPKINS UNIVERSITY, Baltimore, MD (US)

(72) Inventors: Vera IGNJATOVIC, St. Petersburg, FL (US); Neil GOLDENBERG, St. Petersburg, FL (US); Steven BRUZEK, St. Petersburg, FL (US)

(21) Appl. No.: 19/123,531

(22) PCT Filed: Oct. 24, 2023
(86) PCT No.: PCT/US2023/035764

§ 371 Date: Apr. 23, 2025

Related U.S. Application Data
(60) Provisional application No. 63/380,691, filed on Oct. 24, 2022.

(51) Int. Cl. G01N 33/86; G01N 21/31

(52) U.S. Cl. CPC G01N 33/86; G01N 21/31; G01N 2333/7454; G01N 2800/22

(57) ABSTRACT

Provided herein is an automated euglobulin clot lysin assay method for calculating a euglobulin clot lysis time from a plasma sample in an amount of less than about 75 μL. Also disclosed here in a kit analyzing the euglobulin clot lysis time of a plasma sample in an amount of less than about 75 μL, as well as a method for identifying or diagnosing a subject with an abnormal blood condition.

### CROSS-REFERENCE TO RELATED APPLICATION

This application is the national stage entry of International Patent Application No. PCT/US2023/035764, filed on 24 Oct. 2023, and published as WO 2024/091477 A1 on May 2, 2024, which claims the benefit of U.S. Provisional Patent Application No. 63/380,691, filed on 24 Oct. 2022, which are hereby incorporated by reference in their entireties.

### BACKGROUND

Fibrinolysis is the process of dissolving blood clots and is a component of the coagulation system that protects the vasculature from excessive thrombus formation. Alterations in the fibrinolytic system may contribute to abnormal thrombosis or bleeding. A euglobulin clot lysis test was first described in 1948 and is a plasma fibrinolytic test designed to assess how fast clots break down in blood. This test is fully manual and highly subjective, with visual observation of clot formation and lysis used to determine clot formation and lysis times.

A method of using an automated euglobulin clot lysis assay (ECLA) was published in 2003 and allowed for spectrophotometric readings of absorbance to determine lysis times. Smith, A. et al., A new euglobulin clot lysis assay for global fibrinolysis, Thrombosis Res. 2003, 112 (5-6): 329-37. This ECLA is considered the standard screening test for hyper- and hypo-fibrinolytic states. However, the existing ECLA is limited in clinical application due to significant amounts of manual processing, which may lead to low throughput. This has prevented the assay from achieving widespread adoption in coagulation laboratories.

Disclosed herein is a mini-scale ECLA (mini-ECLA) involving significantly less plasma consumption, greater automation, and higher throughout capabilities.

### SUMMARY

Disclosed herein is a mini-scale automated euglobulin clot lysis assay (mini-ECLA) method comprising obtaining a plasma sample from a subject, wherein the plasma sample is in an amount of less than about 75 μL; obtaining a euglobulin fraction pellet from the plasma sample; resuspending the euglobulin fraction pellet in a buffer to create a suspension; adding a calcium/tissue factor protein buffer to the suspension; using a spectrophotometer to determine a median optical density (OD) value and a maximum OD of the suspension; and calculating a euglobulin clot lysis time.

In certain embodiments, the plasma sample is in an amount of less than about 70 μL, such as less than about 65 μL, less than about 60 μL, less than about 55 μL, less than about 50 μL, less than about 45 μL, less than about 40 μL, less than about 35 μL, less than about 30 μL, less than about 25 μL, or about 25 μL, and in certain embodiments, the plasma sample comprises a platelet-poor plasma sample. In certain embodiments, the euglobulin clot lysis time is measured at time intervals from a period of 0 to 10 hours, such as 1 minute to 10 hours or 10 hours, and in certain embodiments, the time intervals range from every 1 minute to every 5 minutes, such as every 3 minutes.

In certain embodiments of the methods disclosed herein, the spectrophotometer is a thermoregulated spectrophotometer, and in certain embodiments, the thermoregulated spectrophotometer is thermoregulated at 37 °C. In certain embodiments, an absorbance of the spectrophotometer for determining the median and maximum ODs is at 405 nm.

In certain embodiments of the methods disclosed herein, the subject is selected from the group consisting of a human, a dog, a cat, a horse, a cow, a sheep, a goat, and a non-human mammal, and in certain embodiments, the subject has or is suspected of having an abnormal blood condition, such as an abnormal blood condition selected from the group consisting of hemophilia, bleeding disorders, and thrombotic disorders. In certain embodiments, the abnormal blood condition is a hyper-fibrinolytic condition, and in certain embodiments, the abnormal blood condition is a hypo-fibrinolytic condition. In certain embodiments, the abnormal blood condition may be a secondary result of a primary condition, such as infection or prior infection of the subject with a virus, e.g., Covid virus.

In certain aspects, the methods disclosed herein further comprise comparing the euglobulin clot lysis time to a second euglobulin clot lysis time obtained from at least one control sample, and in certain aspects, the methods disclosed herein further comprise obtaining a plasma sample from the subject before, during, and/or after treating the subject with at least one therapeutic agent.

In one aspect, the present disclosure provides a kit for analyzing the euglobulin clot lysis time of a plasma sample in an amount of less than about 75 μL comprising a microplate, such as a 96-well microplate; an acetic acid buffer; a sodium borate buffer; tissue factor protein; and a calcium buffer, and optionally instructions for use.

In another aspect, the present disclosure provides a method for identifying or diagnosing a subject with an abnormal blood condition, the method comprising obtaining a plasma sample from the subject, wherein the plasma sample is in an amount of less than about 75 μL; obtaining a euglobulin fraction pellet from the plasma sample; resuspending the euglobulin fraction pellet in a buffer to create a suspension; adding a calcium/tissue factor protein buffer to the suspension; using a spectrophotometer to determine a median optical density (OD) value and a maximum OD of the suspension; calculating a euglobulin clot lysis time for the plasma sample; comparing the euglobulin clot lysis time for the plasma sample to a control euglobulin clot lysis time from a normal plasma sample; and identifying or diagnosing the subject with an abnormal blood condition if the euglobulin clot lysis time for the plasma sample is significantly different from the control euglobulin clot lysis time. In certain embodiments of the method for identifying or diagnosing a subject with an abnormal blood condition, the plasma sample from the subject is in an amount of less than about 70 μL, such as less than about 65 μL, less than about 60 μL, less than about 55 μL, less than about 50 μL, less than about 45 μL, less than about 40 μL, less than about 35 μL, less than about 30 μL, less than about 25 μL, or about 25 μL, and in certain embodiments, the normal plasma sample is in an amount of less than about 70 μL, such as less than about 65 μL, less than about 60 μL, less than about 55 μL, less than about 50 μL, less than about 45 μL, less than about 40 μL, less than about 35 μL, less than about 30 μL, less than about 25 μL, or about 25 μL.

In certain embodiments of the method for identifying or diagnosing a subject with an abnormal blood condition, the abnormal blood condition is a hyper-fibrinolytic condition or a hypo-fibrinolytic condition.

### BRIEF DESCRIPTION OF THE DRAWINGS

The accompanying drawings, which are incorporated in and constitute a part of this specification, illustrate certain embodiments, and together with the written description, serve to explain certain principles of the methods, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1A is a graph showing the average OD measurements over time for the mini-ECLA protocol as described in Example 1 with FACT control, A2AP-deficient, PA1-1 deficient, and plasminogen deficient plasmas, wherein the average 50% clot lysis time±3 standard deviations is marked for each plasma.

FIG. 1B is a graph showing the average OD measurements over time for the standard ECLA protocol as described in Example 1 with FACT control, A2AP-deficient, PA1-1 deficient, and plasminogen deficient plasmas, wherein the average 50% clot lysis time±3 standard deviations is marked for each plasma.

FIG. 2A is a graph showing the average OD measurements over time for the mini-ECLA runs with FACT in the presence of varying concentrations of Tissue Factor addition, showing the ability to generate physiologic clot formation in the mini-ECLA.

FIG. 2B is a graph showing the average OD measurements over time for the mini-ECLA runs with plasminogen-deficient plasma in the presence of varying concentrations of Tissue Factor addition, showing the ability to generate physiologic clot formation in the mini-ECLA.

FIG. 3A is a graph showing the average OD measurements over time for concentration ranges of PAI-1 deficient plasma, wherein the average 50% clot lysis time±3 standard deviations is marked for each plasma concentration. The plot is normalized to clot formation at 0 minutes for visual comparison of clot lysis times.

FIG. 3B is a graph showing the average OD measurements over time for concentration ranges of A2AP deficient plasma, wherein the average 50% clot lysis time±3 standard deviations is marked for each plasma concentration. The plot is normalized to clot formation at 0 minutes for visual comparison of clot lysis times.

FIG. 3C is a graph showing the average OD measurements over time for concentration ranges of plasminogen-deficient plasma, wherein the average 50% clot lysis time±3 standard deviations is marked for each plasma concentration. The plot is normalized to clot formation at 0 minutes for visual comparison of clot lysis times.

FIG. 3D is a graph showing the average OD measurements over time for concentration ranges of excess tPA plasma, wherein the average 50% clot lysis time #3 standard deviations is marked for each plasma concentration. The plot is normalized to clot formation at 0 minutes for visual comparison of clot lysis times.

FIG. 4A is a graph showing the average OD measurements over time for regular (non-mini) ECLA runs with FACT and samples 100% deficient in plasminogen, PAI-1, and A2AP. Error bars represent±1 S.D. of the mean adjusted Clot Lysis Time at the median OD value when applicable.

FIG. 4B is a graph showing the average OD measurements over time for mini-ECLA runs with FACT and sample 100% deficient in plasminogen, PAI-1, and A2AP. Error bars represent #1 S.D. of the mean adjusted Clot Lysis Time at the median OD value when applicable.

FIG. 4C is a graph showing the average OD measurements over time for mini-ECLA runs for either 0 μM or 1 μM of relipidated tissue factor together with either 0 ng/mL or 0.7 ng/ml of tissue plasminogen activator.

FIG. 4D is a graph showing a representative curve of a mini-ECLA run showing the main data outputs, including maximum OD (clot formation), median OD (50% clot lysis), area under the curve (AUC), and aCLT.

FIG. 5A is a graph showing the average OD measurements over time for mini-ECLA runs with samples (n=5) of 100%, 70%, 60%, 50%, 40%, and 30% fibrinogen.

FIG. 5B is a graph showing the change in the average OD over time for mini-ECLA runs with samples (n=5) of 100%, 70%, 60%, 50%, 40%, and 30% fibrinogen; 95% confidence interval (CI) shown.

FIG. 5C is a graph showing the AUC over time for mini-ECLA runs with samples (n=5) of 100%, 70%, 60%, 50%, 40%, and 30% fibrinogen; 95% CI shown.

FIG. 5D is a graph showing the clot lysis time ratio (CLTR) over time for mini-ECLA runs with samples (n=5) of 100%, 70%, 60%, 50%, 40%, and 30% fibrinogen; 95% CI shown.

FIG. 5E is a graph showing the CLTR OD adjusted over time for mini-ECLA runs with samples (n=5) of 100%, 70%, 60%, 50%, 40%, and 30% fibrinogen; 95% CI shown.

FIG. 6A is a graph showing the average OD measurements over time for mini-ECLA runs with FACT and samples (n=8) of plasma 100% deficient in plasminogen, PAI-1, and A2AP.

FIG. 6B is a graph showing the average OD measurements over time for mini-ECLA runs with FACT and samples (n=8) of plasma 75% deficient in plasminogen, PAI-1, and A2AP.

FIG. 6C is a graph showing the average CLTR for each of plasminogen-deficient plasma, PAI-1 deficient plasma, and A2AP deficient plasma at 100% (n=8), 75% (n=8), 50% (n=3), and 25% (n=3) deficiency; 95% CI shown. A horizontal bar at 1.0 represents normal CLTR in FACT plasma.

FIG. 6D is a graph showing the average CLTR OD adjusted for each of plasminogen-deficient plasma, PAI-1 deficient plasma, and A2AP deficient plasma at 100% (n=8), 75% (n=8), 50% (n=3), and 25% (n=3) deficiency; 95% CI shown. A horizontal bar at 1.0 represents normal CLTR OD Adjusted in FACT plasma.

FIG. 7A are box and whisker plots showing CLTR values for convalescent, critically ill, and Covid patient plasma as determined using a mini-ECLA.

FIG. 7B are box and whisker plots showing fibrinolytic indices (FI2%) for convalescent, critically ill, and Covid patient plasma as determined using a CloFAL turbidimetric assay.

FIG. 7C is a plot showing the fibrinolytic indices FI2% plotted against the corresponding CLTR values for convalescent, critically ill, and Covid patient plasma, showing the correlation coefficient (r) and p value of the correlation coefficient.

FIG. 8A are box and whisker plots showing CLTR OD Adjusted values for convalescent, critically ill, and Covid patient plasma as determined using a mini-ECLA.

FIG. 8B are box and whisker plots showing fibrinolytic indices (FI2%) for convalescent, critically ill, and Covid patient plasma as determined using a CloFAL turbidimetric assay.

FIG. 8C is a plot showing the fibrinolytic indices FI2% plotted against the corresponding CLTR OD Adjusted values for convalescent, critically ill, and Covid patient plasma, showing the correlation coefficient (r) and p value of the correlation coefficient.

FIG. 9A are box and whisker plots showing the change in OD ratio, or delta OD ratio (DODR) values for convalescent, critically ill, and Covid patient plasma as determined using a mini-ECLA.

FIG. 9B are box and whisker plots showing coagulation indices (CI %) for convalescent, critically ill, and Covid patient plasma as determined using a CloFAL turbidimetric assay.

FIG. 9C is a plot showing the coagulation indices CI % plotted against the corresponding DODR values for convalescent, critically ill, and Covid patient plasma, showing the correlation coefficient (r) and p value of the correlation coefficient.

FIG. 10A are box and whisker plots showing the area under the curve ratio (AUCR) for convalescent, critically ill, and Covid patient plasma as determined using a mini-ECLA.

FIG. 10B are box and whisker plots showing AUC % for convalescent, critically ill, and Covid patient plasma as determined using a CloFAL turbidimetric assay/

FIG. 10C is a plot showing the AUC % plotted against the corresponding AUCR values for convalescent, critically ill, and Covid patient plasma, showing the correlation coefficient (r) and p value of the correlation coefficient.

### DETAILED DESCRIPTION

Disclosed herein is an automated euglobulin clot lysis assay (ECLA) and methods of using the same. In the ECLA disclosed herein, there is a series of acidification, centrifugation, and wash steps, followed by resuspension of the resulting euglobulin fraction pellet in preparation for absorbance readings. In certain embodiments, the absorbance readings are performed at about 405 nm about every 3 minutes for up to about 10 hours in a thermoregulated spectrophotometer at about 37 °C.

Total patient plasma that may be used for the assay disclosed herein is less than about 70 μL, such as about 50 μL. A mini-ECLA clot lysis time may be calculated as the time the absorbance reaches the median OD value between the minimum and maximum OD readings (50% clot lysis), minus the time at maximum OD reading (full clot formation). The methods disclosed herein retain the use of pooled normal plasma standards and both hyperfibrolytic and hypofibrolytic controls.

In an embodiment disclosed herein, there is a 96-well microplate to which 300 μL of cold, 0.02% acetic acid is added to 25 μL of platelet-poor plasma in duplicate for each specimen. After a 10 minute incubation on wet ice, the plate is spun at 2000×g at 25 °C for 5 minutes. The supernatant is removed by plate inversion, and 100 μL of Milli-Q water is added to each well. The plate is spun at 2000×g at 25 °C for 5 minutes, and the supernatant is again removed by plate inversion. The euglobulin fraction pellet is resuspended in 100 μL of pre-warmed (37 °C) sodium borate buffer, mixed by shaking, and incubated at 37 °C for 3 minutes. To one of the duplicates, 100 μL of pre-warmed (37 °C) calcium buffer is added. The remaining well is used as a negative control. The plate reader protocol comprises absorbance readings at 405 nm every 3 minutes for 10 hours, with an initial orbital shaking step and incubation at 37 °C for the duration of the run. Mini-ECLA clot lysis time is calculated as the time the absorbance reaches the median OD value between the minimum and maximum OD readings (50% clot lysis), minus the time at maximum OD reading (full clot formation).

The ability to perform the entirety of the methods disclosed herein the 96-well plate dramatically improves throughput. Additionally, the addition of tissue factor to calcium buffer may generate physiologic conditions of clot formation and reduce previous issues of reduced clot formation in mini-volume processing. As compared to previous methods, changes in proportions/ratios of plasma to buffers disclosed herein may generate a robust signal in mini-scale version without sacrificing sensitivity to protein-deficient states.

### EXAMPLE

### Example 1—Mini-Scale ECLA

Methods: To a 96-well microplate, 300 μL of cold, 0.02% acetic acid was added to 25 μL of platelet-poor plasma in duplicate for each specimen. After a 10 minute incubation on wet ice, the plate was spun at 2000×g at 25 °C for 5 minutes. The supernatant was removed by plate inversion, and 100 μL of Milli-Q water was added to each well. The plate was spun at 2000×g at 25 °C for 5 minutes, and the supernatant was again removed by plate inversion. The euglobulin fraction pellet was resuspended in 100 μL of pre-warmed (37 °C) sodium borate buffer, mixed by shaking, and incubated at 37 °C for 3 minutes. To one of the duplicates, 100 μL of pre-warmed (37 °C) calcium buffer was added. The remaining well was used as a negative control. Absorbance readings were conducted at 405 nm every 3 minutes for 10 hours, with an initial orbital shaking step and incubation at 37 °C for the duration of the run. Mini-ECLA clot lysis time was calculated as the time the absorbance reaches the median OD value between the minimum and maximum OD readings (50% clot lysis), minus the time at maximum OD reading (full clot formation).

Table 1 below illustrates the methods disclosed herein as compared to a standard ECLA method.

TABLE 1
Exemplary Mini-ECLA Methods

| Protocol step | Mini-ECLA | |
|---|---|---|
| Plasma volume | 50 μL | 350 μL |
| Euglobulin | 25 μL plasma + | 350 μL plasma + |
| Fraction | 300 μL 0.02% | 6.3 mL 0.02 acetic |
| Generation | acetic acid (1:13); | acid (1:19); |
| | Incubate in 96-well plate for | Incubate in 10 mL |
| | 10 minutes on wet ice | glass vial for 10 |
| | | minutes on wet ice |
| Euglobulin | Spin at 2000 × g for 5 min, | Spin at 2000 × g for |
| Fraction | remove supernatant by | 5 min, remover |
| Concentration | inversion; | supernatant by |
| | Wash with 100 μL Milli-Q | inversion |
| | water; | |
| | Spin at 2000 × g for 5 min, | |
| | remove supernatant by | |
| | inversion | |
| Resuspension | Resuspend with 100 μL | Resuspend with |
| | sodium borate buffer (1:4 | 350 μL sodium borate |
| | plasma to buffer) directly in | buffer (1:1 plasma to |
| | sample well | buffer), split into 2 × |
| | | 150 μL aliquots for |
| | | each sample |
| Clot | Add 1:1 calcium buffer with | Add 1:1 calcium |
| Formation | addition of tissue factor to | buffer to sample |
| | sample | |

Results: Clot lysis times in mini-ECLA were determined in a representative sample of commercial control plasmas, as follows: Factor Assay Control Plasma (FACT) (George King Biomedical, 0020-0), 276±15 minutes (n=10); Pooled Normal Plasma (PNP) (George King Biomedical, 0010-0), 306±14 minutes (n=10). Representative samples of commercial plasminogen deficient (Affinity Biologicals, PG-DP), alpha-2-antiplasmin deficient [Affinity Biologicals, A2AP-DP), and plasminogen activator inhibitor-1 (PAI-1) deficient (Affinity Biologicals, PAI-DP) plasmas were run with preliminary data showing hypofibrinolysis for PAI-1 and plasminogen deficient plasmas with average clot lysis times to be determined. The results are shown below in Table 2. Clot lysis times (50%) used to determine diagnostic ability of mini-ECLA and ECLA methods were calculated as the average clot lysis time falling outside of 3 standard deviations from the average clot lysis time of FACT.

TABLE 2
Comparison of Mini-ECLA and ECLA Clot Lysis Times

| | Mini-ECLA | | ECLA (Smith 2003) | |
|---|---|---|---|---|
| | 50% Clot Lysis | Potential | 50% Clot Lysis | Potential |
| | Time | Diagnostic | Time | Diagnostic |
| FACT | 314 ± 7.9 min | | 323 ± 12.6 | |
| | (n = 20) | | (n = 6) | |
| 100% PAI-1 | 528 ± 16.4 min | Yes - | 490 ± 24.4 min | Yes - |
| Deficient | (n = 5) | hypofibrinolysis | (n = 3) | hypofibrinolysis |
| 100% A2AP | 280 ± 4.8 min | Yes - | 328.5 ± 10.6 min | No |
| Deficient | (n = 13) | hyperfibrinolysis | (n = 2) | |
| 100% | >600 min | Yes - | >600 min | Yes - |
| Plasminogen | (n = 3) | hypofibrinolysis | (n = 2) | hypofibrinolysis |
| Deficient | | | | |
| Excess tPA | 18 ± 0 min | Yes - | 45 min | Yes - |
| | (n = 4) | hyperfibrinolysis | (n = 1) | hyperfibrinolysis |

Conclusions: The results demonstrate the development of a mini-ECLA as a modification of the original ECLA, showing preliminary analytic sensitivity to multiple alterations in fibrinolytic states while allowing for significant sample preservation, greater automation, and higher throughput. On-going efforts are focused on establishing coefficients of variation and further evaluating analytic sensitivity to known regulators of fibrinolysis.

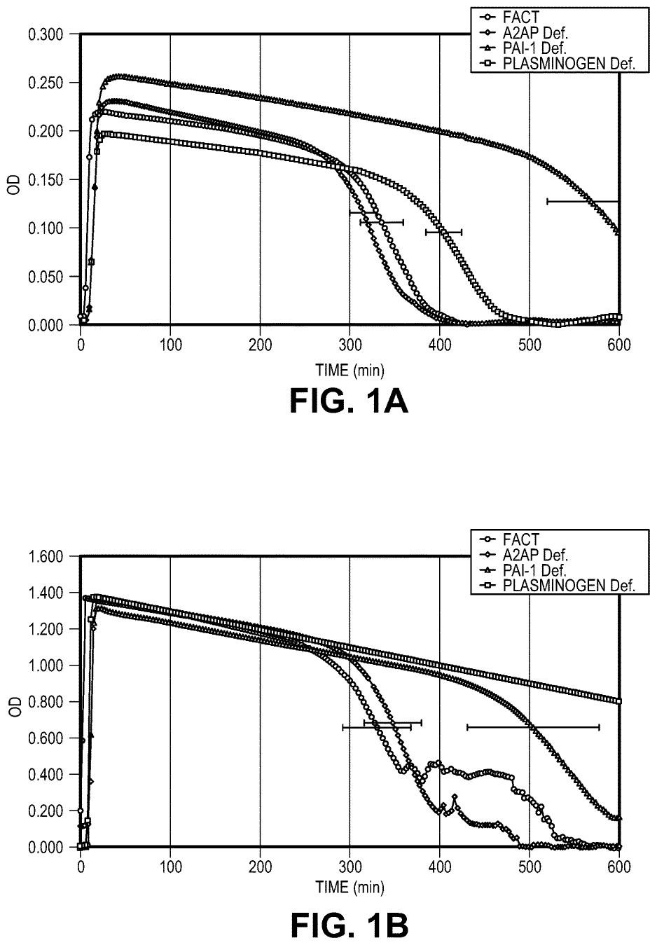

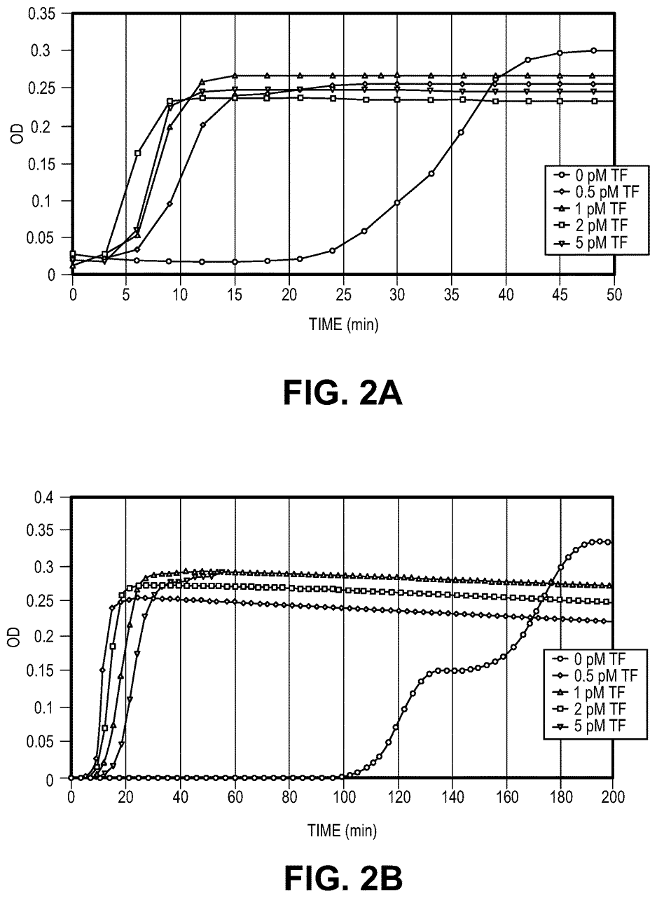

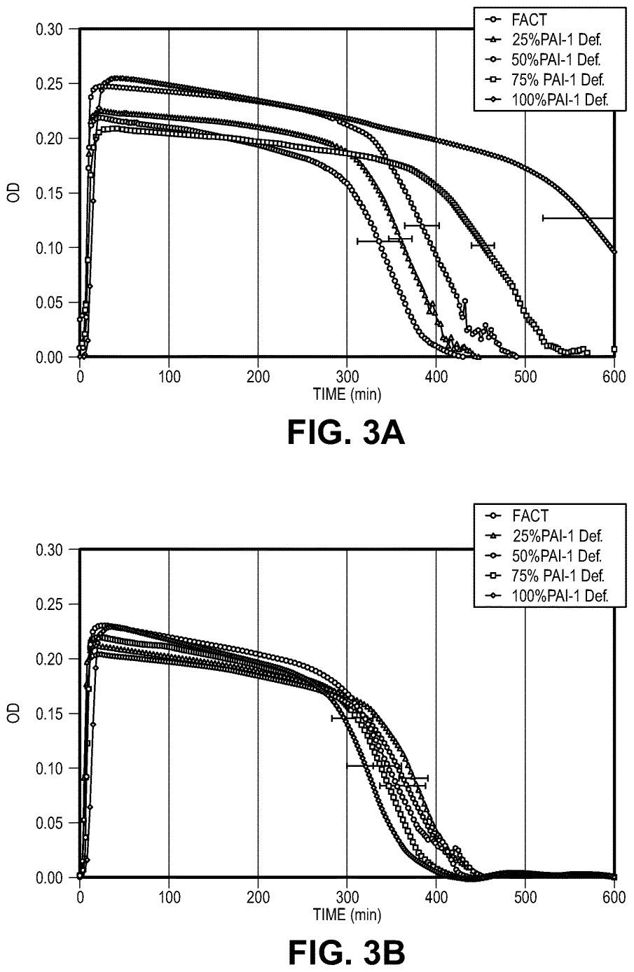

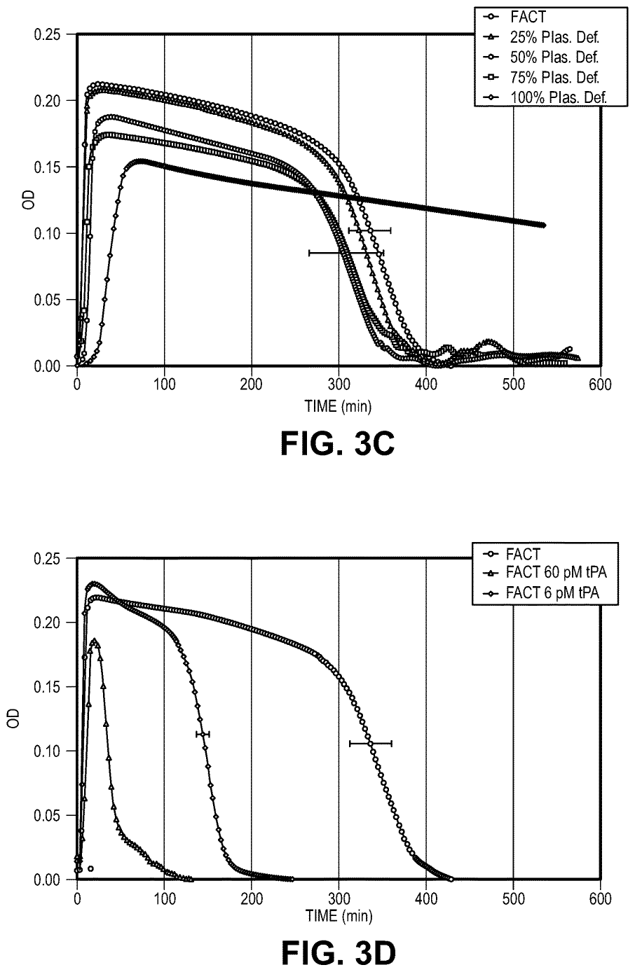

As shown in the FIGS. 1A-1B, the mini-ECLA method is concordant with a standard ECLA method for each of the FACT control, A2AP-deficient plasma, PAI-1 deficient plasma, and plasminogen-deficient plasma. As shown in FIGS. 2A-2B, the mini-ECLA methods disclosed herein are capable of generating physiologic clot formation in the presence of Tissue Factor. FIGS. 3A-3D illustrate that the mini-ECLA methods disclosed herein may be diagnostic for all deficient plasma states relative to data from existing standard ECLA methods.

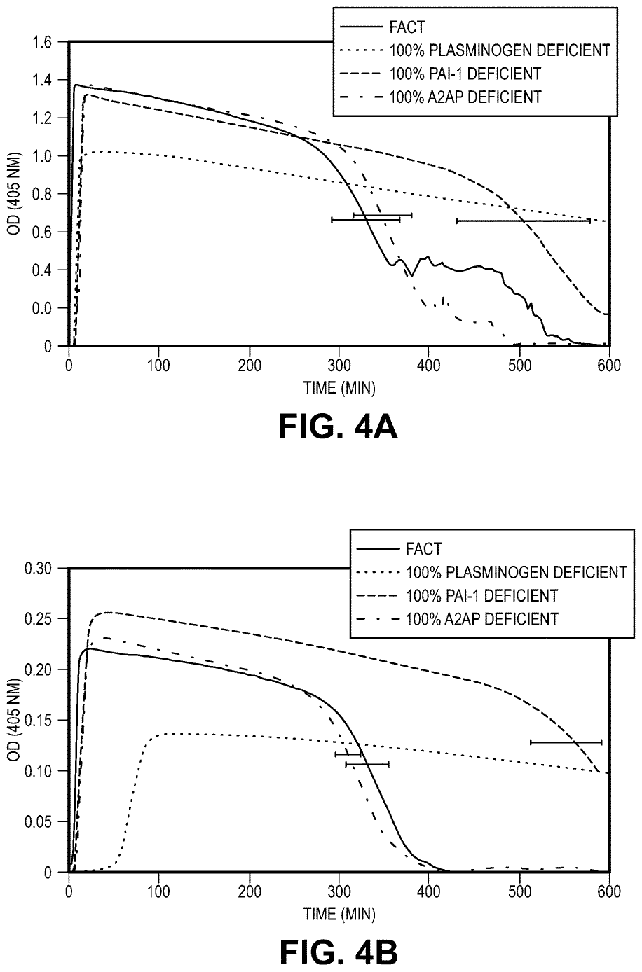

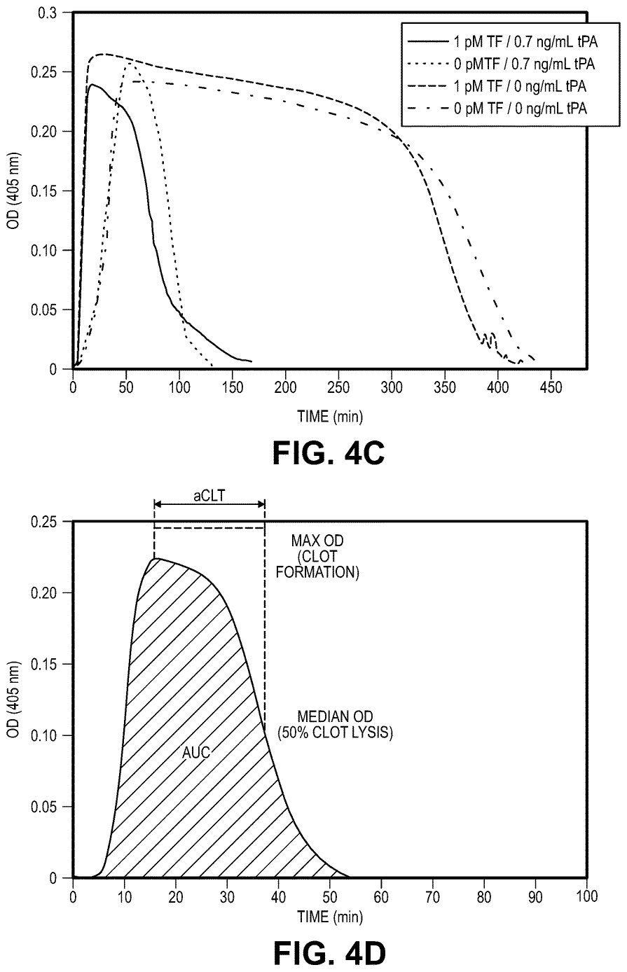

FIG. 4A shows a clot lysis tracing of an original ECLA method with FACT and 100% deficient plasma samples. In contrast, FIG. 4B shows a clot lysis tracing of a volume reduction mini-ECLA with FACT and 100% deficient plasma samples. In both FIGS. 4A and 4B, where applicable, error bars show+/−SD of the mean adjusted Clot Lysis Time at the median OD value. As shown in FIG. 4B, the results are concordant to the results of the original ECLA method. FIG. 4C shows a clot lysis tracing of the modified mini-ECLA final configuration development through the addition of relipidated tissue factor (TF) and tissue plasminogen activator (tPA), and FIG. 4D shows a representative clot lysis tracing of the modified mini-ECLA disclosed herein, showing exemplary main data outputs.

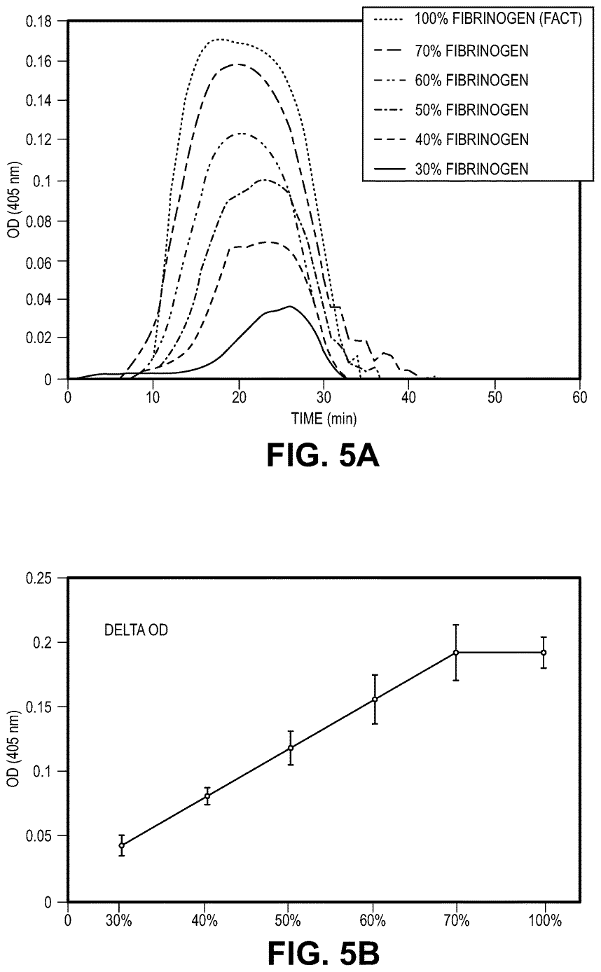

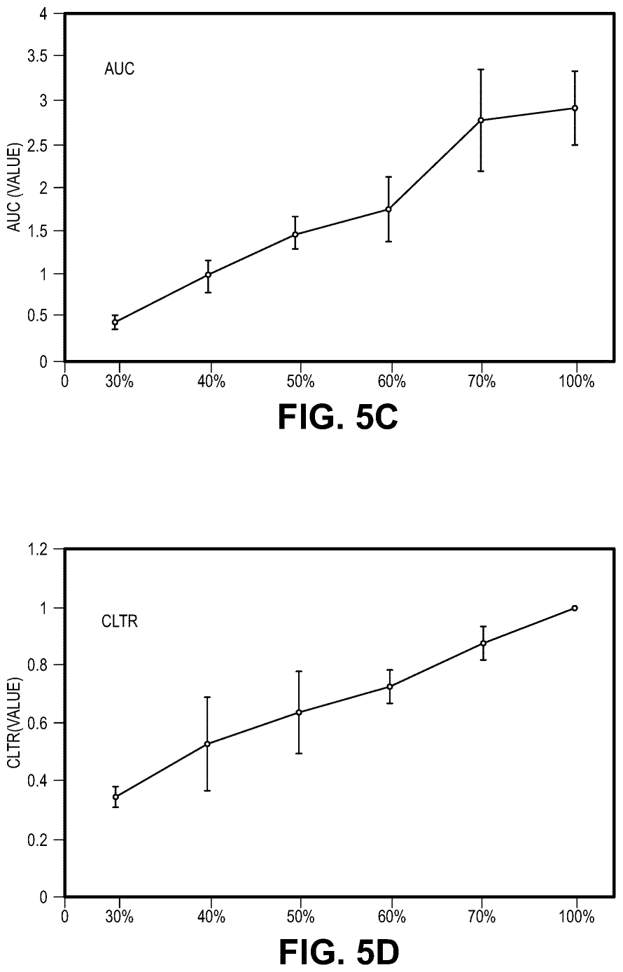

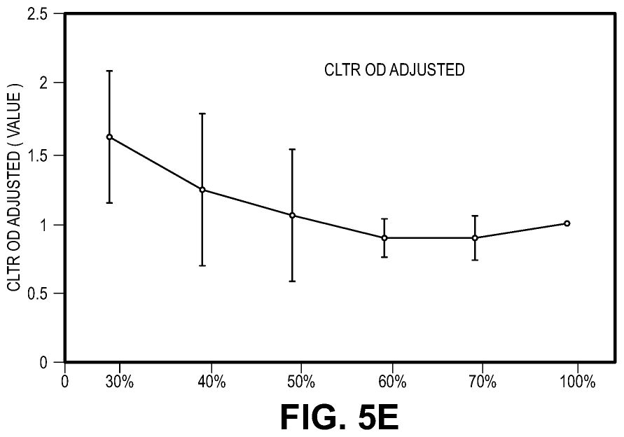

FIG. 5A shows a fibrinogen concentration curve for various percent fibrinogen samples of plasma, from 100% (FACT) to 30% fibrinogen, using a modified mini-ECLA process as disclosed herein. In FIG. 5A, the average clot lysis curves of contrived samples (n=5) were created by mixing 100% fibrinogen deficient plasma with FACT, such that 100% fibrinogen=290 mg/dL; 70% fibrinogen=203 mg/dL, 60% fibrinogen=174 mg/dL; 50% fibrinogen-145 mg/dL; 40% fibrinogen=116 mg/dL; and 30% fibrinogen=87 mg/dL. FIG. 5B does the delta OD average (n=5) for each of the 6 fibrinogen levels with a 95% confidence interval shown, and FIG. 5C shoes the area under the curve (AUC) for each of the 6 fibrinogen levels with a 95% confidence interval shown. FIG. 5D shows the clot lysis time ratio (CLTR) for each of the 6 fibrinogen levels with a 95% confidence interval shown, and FIG. 5E shows the clot lysis time ratio OD adjusted (CLTR OD Adjusted) for each of the 6 fibrinogen levels with a 95% confidence interval shown.

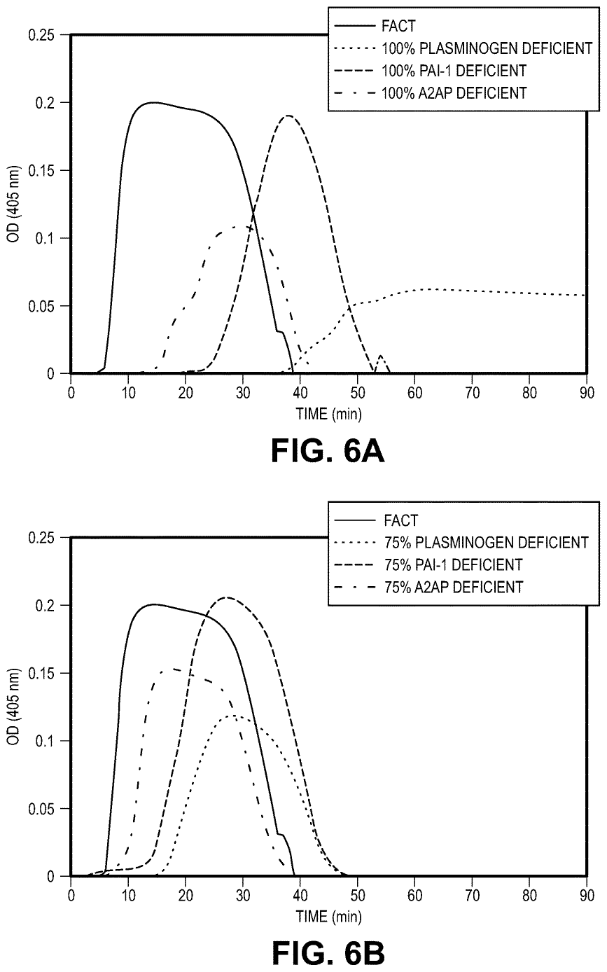

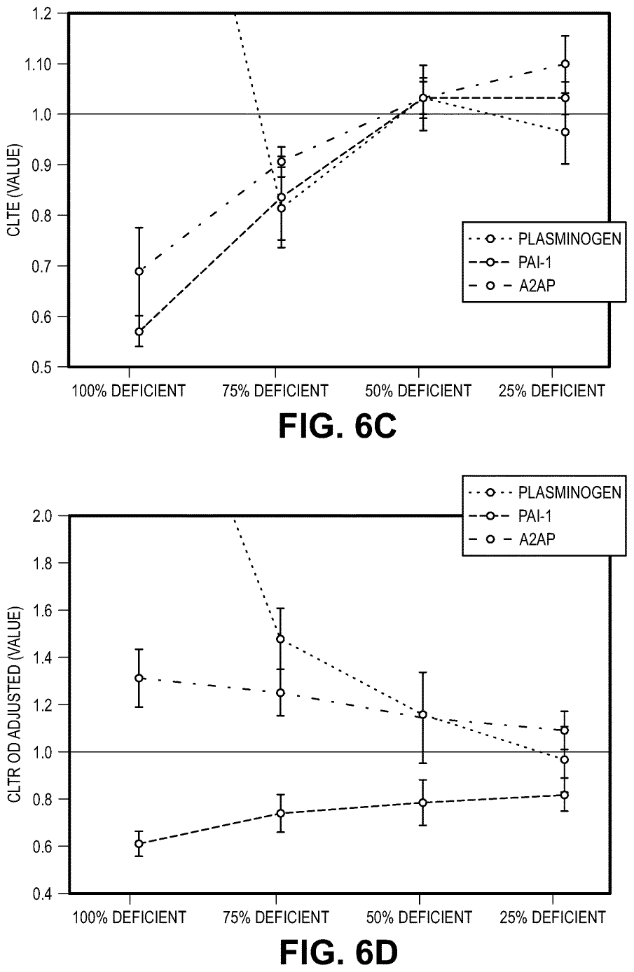

In FIG. 6A, plasma tracings using the modified mini-ECLA process disclosed herein are shown of an average (n=8) of 100% deficient commercial control plasmas, including 100% plasminogen deficient, 100% PAI-1 deficient, and 100% A2AP deficient, as well as a FACT control. FIG. 6B shows plasma tracings using the modified mini-ECLA process disclosed herein are shown of an average (n=8) of 75% deficient commercial control plasmas (spiked with 25% FACT), including 75% plasminogen deficient, 75% PAI-1 deficient, and 75% A2AP deficient, as well as a FACT control. FIG. 6C shows the average CLTR for each of the three deficient plasma types (plasminogen deficient, PAI-1 deficient, and A2AP deficient) across multiple levels of deficiency (100% deficient (n=8), 75% deficient (n=8), 50% deficient (n=3), and 25% deficient (n=3)) generated by spiking 100% deficient plasma with FACT control plasma. FIG. 6D shows the average CLTR OD Adjusted for each of the deficient plasma types shown in FIG. 6C. In FIG. 6D, error bars show 95% confidence interval, and the horizontal bar at 1.0 represents a normal CLTR OD Adjusted in FACT plasma, with CLTR OD Adjusted values lower than 1.0 representing hyperfibrinolysis and CLTR OD Adjusted values above 1.0 representing hypofibrinolysis. The averages from FIG. 6C and FIG. 6D are shown below in Table 2. In Table 2 below, P values were determined by a nonpaired Student's t-test reported as ns=not significant, *p<0.05, **p<0.01, ***p<0.001.

TABLE 2
Modified mini-ECLA CLTR and CLTR OD Adjusted
values for deficient plasma samples

| | CLTR | | CLTR OD Adjusted | |
|---|---|---|---|---|
| Plasma Sample | (Average ± S.D.) | | (Average ± S.D.) | |
| 100% Plasminogen Deficient | 10.8 ± 1.53 | (n = 8) *** | 41.17 ± 14.96 | (n = 8) *** |
| 75% Plasminogen Deficient | 0.82 ± 0.11 | (n = 8) *** | 1.48 ± 0.18 | (n = 8) *** |
| 50% Plasminogen Deficient | 1.03 ± 0.04 | (n = 3) ^{ns} | 1.15 ± 0.01 | (n = 3) ^{ns} |
| 25% Plasminogen Deficient | 0.97 ± 0.06 | (n = 3) ^{ns} | 0.97 ± 0.12 | (n = 3) ^{ns} |
| 100% PAI-1 Deficient | 0.57 ± 0.08 | (n = 24) *** | 0.61 ± 0.08 | (n = 24) *** |
| 75% PAI-1 Deficient | 0.83 ± 0.12 | (n = 8) ** | 0.74 ± 0.11 | (n = 8) *** |
| 50% PAI-1 Deficient | 1.03 ± 0.03 | (n = 3) ^{ns} | 0.78 ± 0.09 | (n = 3) * |
| 25% PAI-1 Deficient | 1.03 ± 0.03 | (n = 3) ^{ns} | 0.82 ± 0.06 | (n = 3) * |
| 100% A2AP Deficient | 0.69 ± 0.12 | (n = 8) *** | 1.31 ± 0.18 | (n = 8) ** |
| 75% A2AP Deficient | 0.91 ± 0.04 | (n = 8) * | 1.25 ± 0.14 | (n = 8) ** |
| 50% A2AP Deficient | 1.03 ± 0.06 | (n = 3) ^{ns} | 1.14 ± 0.17 | (n = 3) ^{ns} |
| 25% A2AP Deficient | 1.10 ± 0.05 | (n = 3) * | 1.09 ± 0.07 | (n = 3) ^{ns} |

### Example 2—Mini-ECLA to Evaluate Convalescent, Critically III, and Covid Patient Plasma

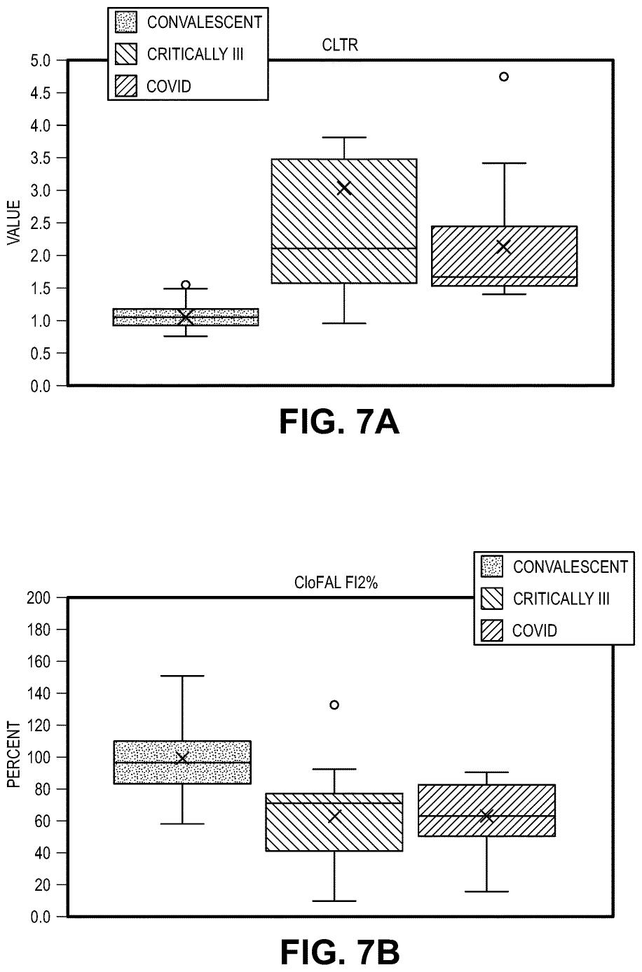

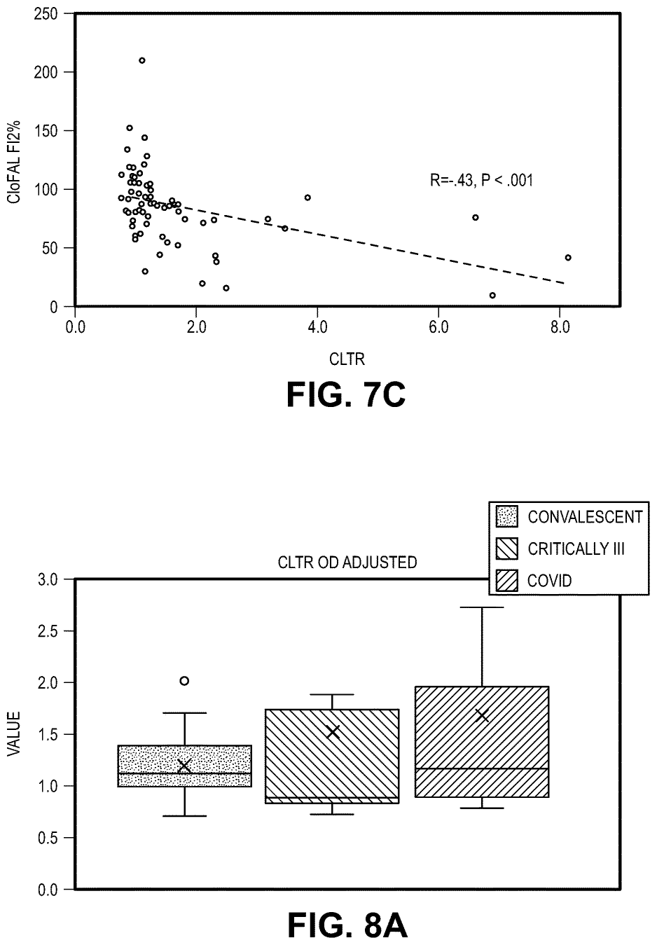

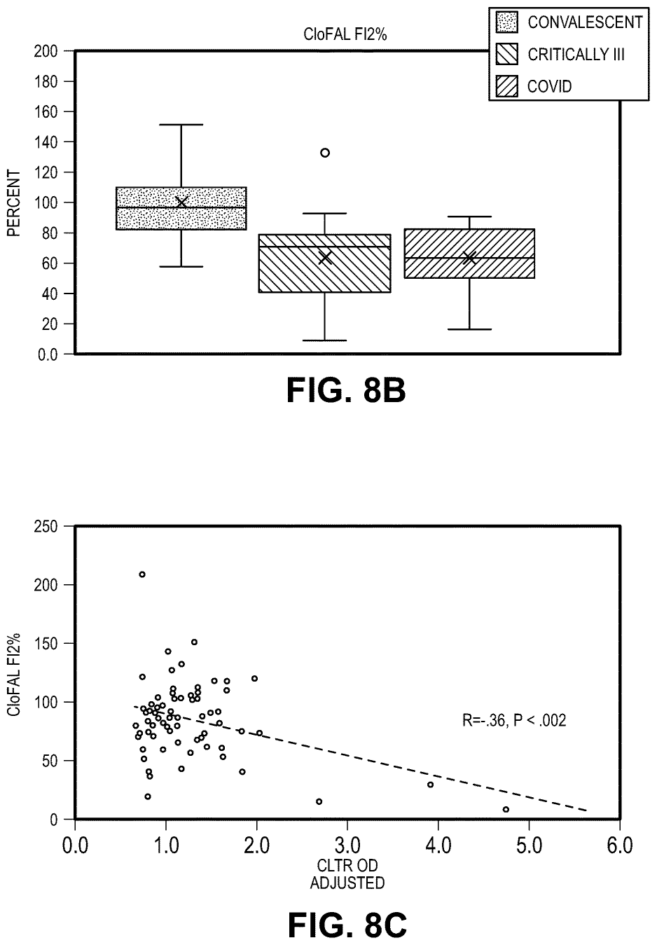

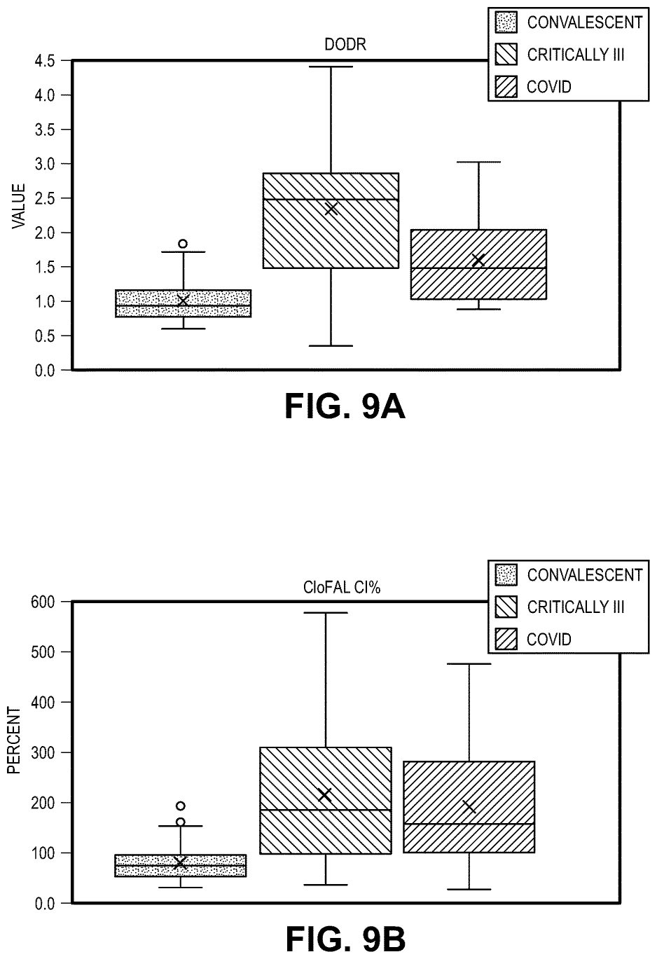

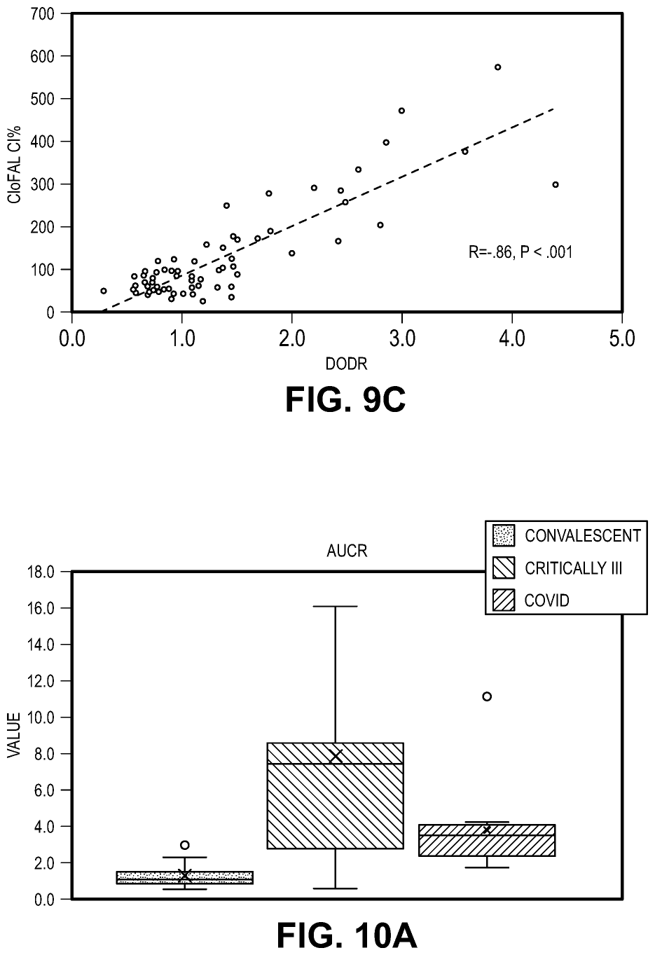

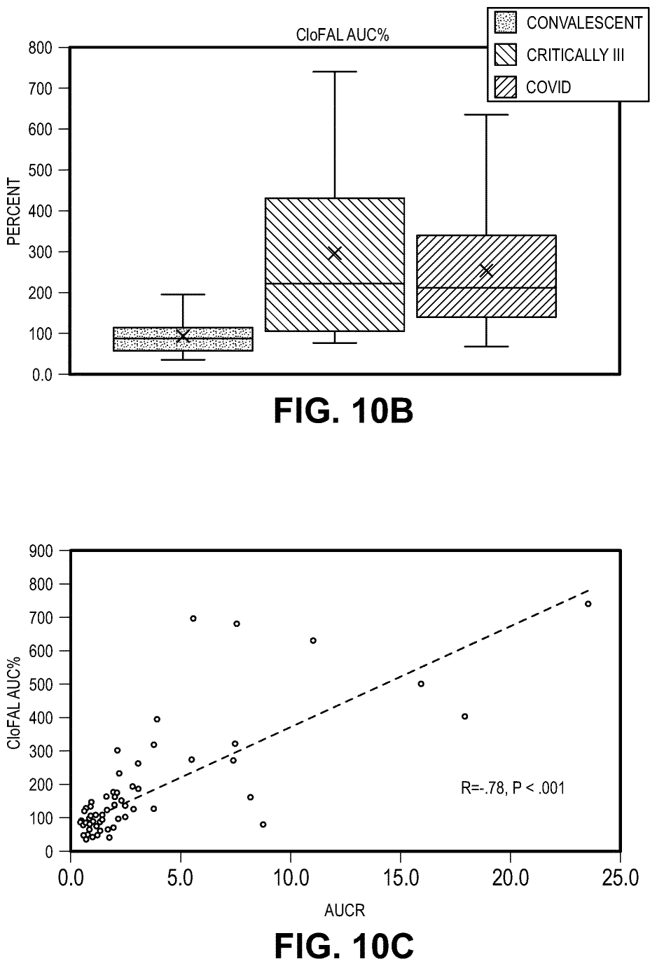

Modified-ECLA and CloFAL metrics were compared in plasma samples obtained from convalesced patients (3 month post-VTE, n=48), critically ill patients (24-48 hours post-invasively ventilated in ICU, n=15), and Covid patients (on day 0 of admission for confirmed Covid-19, n=12). FIG. 7A shows the CLTR from a modified mini-ECLA process as disclosed herein for each of the three patient populations, while FIG. 7B shows the CloFAL FI2% (fibrinolytic index) for each of the three patient population and FIG. 7C shows a plot of the CLTR plotted against the corresponding CloFAL FI2%, with the correlation coefficient (r) and p value of correlation coefficient shown as plot inset. FIG. 8A shows the CLTR OD Adjusted for each of the patient populations, while FIG. 8B shows the CloFAL FI2% and FIG. 8C shows a plot of the CLTR OD Adjusted plotted against the corresponding CloFAL FI2%, with the correlation coefficient (r) and p value of correlation coefficient shown as plot inset. FIG. 9A shows the delta OD ratio (DODR) for each of the patient populations, while FIG. 9B shows the CloFAL CI % (coagulation index), and FIG. 9C shows a plot of the DODR plotted against the corresponding CloFAL CI %, with the correlation coefficient (r) and p value of correlation coefficient shown as plot inset. Finally, FIG. 10A shows the area under the curve ratio (AUCR) for each of the patient populations, while FIG. 10B shows the CloFAL AUC %, and FIG. 10C shows a plot of the AUCR plotted against the corresponding CloFAL AUC %, with the correlation coefficient (r) and p value of correlation coefficient shown as plot inset.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, devices, systems, computer readable media, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

What is claimed is:

1. A mini-scale automated euglobulin clot lysis assay (mini-ECLA) method comprising:
obtaining a plasma sample from a subject, wherein the plasma sample is in an amount of less than about 75 μL;
obtaining a euglobulin fraction pellet from the plasma sample;
resuspending the euglobulin fraction pellet in a buffer to create a suspension;
adding a calcium/tissue factor protein buffer to the suspension;
using a spectrophotometer to determine a median optical density (OD) value and a maximum OD of the suspension; and
calculating a euglobulin clot lysis time.

2. The method of claim 1, wherein the plasma sample is in an amount of less than about 70 μL, such as less than about 65 μL, less than about 60 μL, less than about 55 μL, less than about 50 μL, less than about 45 μL, less than about 40 μL, less than about 35 μL, less than about 30 μL, less than about 25 μL, or about 25 μL.

3. The method of claim 1, wherein the plasma sample comprises a platelet-poor plasma sample.

4. The method of claim 1, wherein the euglobulin clot lysis time is measured at time intervals from a period of 0 to 10 hours.

5. The method of claim 4, wherein the period is 10 hours.

6. The method of claim 4, wherein the time intervals range from every 1 minute to every 5 minutes, such as every 3 minutes.

7. The method of claim 1, the spectrophotometer is a thermoregulated spectrophotometer at 37 °C.

8. The method of claim 1, wherein an absorbance of the spectrophotometer for determining the median and maximum ODs is at 405 nm.

9. The method of claim 1, wherein the subject is selected from the group consisting of a human, a dog, a cat, a horse, a cow, a sheep, a goat, and a non-human mammal.

10. The method of claim 1, wherein the subject has or is suspected of having an abnormal blood condition.

11. The method of claim 10, wherein the abnormal blood condition is selected from the group consisting of hemophilia, bleeding disorders, and thrombotic disorders.

12. The method of claim 11, wherein the abnormal blood condition is a hyper-fibrinolytic condition.

13. The method of claim 11, wherein the abnormal blood condition is a hypo-fibrinolytic condition.

14. The method of claim 1, further comprising comparing the euglobulin clot lysis time to a second euglobulin clot lysis time obtained from at least one control.

15. The method of claim 1, further comprising obtaining a plasma sample from the subject before, during, and/or after treating the subject with at least one therapeutic agent.

16. A kit for analyzing the euglobulin clot lysis time of a plasma sample in an amount of less than about 75 μL comprising:
a microplate;
a sodium borate buffer;
tissue factor protein; and
a calcium buffer.

17. The kit of claim 16, wherein the microplate is a 96-well microplate.

18. A method for identifying or diagnosing a subject with an abnormal blood condition, the method comprising:
obtaining a plasma sample from the subject, wherein the plasma sample is in an amount of less than about 75 μL;
obtaining a euglobulin fraction pellet from the plasma sample;
resuspending the euglobulin fraction pellet in a buffer to create a suspension;
adding a calcium/tissue factor protein buffer to the suspension;
using a spectrophotometer to determine a median optical density (OD) value and a maximum OD of the suspension;
calculating a euglobulin clot lysis time for the plasma sample;
comparing the euglobulin clot lysis time for the plasma sample to a control euglobulin clot lysis time from a normal plasma sample; and
identifying or diagnosing the subject with an abnormal blood condition if the euglobulin clot lysis time for the plasma sample is significantly different from the control euglobulin clot lysis time.

19. The method of claim 18, wherein the plasma sample is in an amount of less than about 70 μL, such as less than about 65 μL, less than about 60 μL, less than about 55 μL, less than about 50 μL, less than about 45 μL, less than about 40 μL, less than about 35 μL, less than about 30 μL, less than about 25 μL, or about 25 μL.

20. The method of claim 18, wherein the abnormal blood condition is a hyper-fibrinolytic condition or a hypo-fibrinolytic condition.
